(19) United States
(12) Patent Application Publication
(10) Pub. No.: US 2022/0241160 A1
(43) Pub. Date: Aug. 4, 2022

(54) Assembled Jade Activation Material for Beauty and Assembly and Synthesis Method and Application thereof

(71) Applicant: SICHUAN SANTAI PHARMACEUTICAL TECHNOLOGY CO. LTD, Chengdu, Sichuan (CN)

(72) Inventors: Haili GAO, Chengdu, Sichuan (CN); Ling WANG, Chengdu, Sichuan (CN); Maokui HE, Chengdu, Sichuan (CN)

(21) Appl. No.: 17/596,559

(22) PCT Filed: Apr. 30, 2020
(86) PCT No.: PCT/CN2020/088209

§ 371 Date: Dec. 13, 2021

(51) Int. Cl. A61K 8/27; A61K 8/23; A61Q 19/08; A61K 8/02

(52) U.S. Cl. CPC A61K 8/27; A61K 8/23; A61K 2800/805; A61K 8/0225; A61Q 19/08

(57) ABSTRACT

An assembled jade activation material is assembled and synthesized by the sol-gel method, and contains 55-59% tetraethyl orthosilicate, 4-8% calcium nitrate tetrahydrate, 26-30% magnesium nitrate hexahydrate, 1-8% zinc nitrate, 0-3% yeast selenium, 0-1% manganese nitrate and 0-1% copper nitrate by molar ratio, and the sum of the mole percentages of each component is 100%. The assembly method draws on the main components of natural jade materials that exert their effects, and synthesizes the assembling jade activation materials through self-assembly of sol-gel technology, uniquely increases silicon, calcium, magnesium ions, etc., and adjusts the amount of trace elements according to needs, adding zinc, selenium, manganese, copper and other elements.

### TECHNICAL FIELD

The invention belongs to the technical field of beauty and skin care materials, and more specifically, relates to an assembled jade activation material for beauty and an assembly and synthesis method thereof.

### BACKGROUND ART

Since ancient times, jade materials have been used for beauty, nourishing and skin care. Jade materials as the original material of female makeup and beauty. According to the classic Chinese medicine “Compendium of Materia Medica” records, jade has the effect of clearing heat and detoxifying, moisturizing skin and producing muscle, activating blood and collaterals, and waking the brain in the eyes. The value of jade in medicine is also highly appraised in the Book of Rites. Jade material also has anti-aging, improve skin microcirculation, improve skin immunity, repair skin damage, anti-inflammatory and other effects.

In recent years, although the application of jade materials in the field of beauty and skin care has been involved, the existing products are all natural jade materials. The composition of natural jade materials fluctuates greatly, and the composition of natural jade materials from different origins is not exactly the same. The composition of natural jade is limited and screening is difficult. Natural jade generally lacks sufficient or over-proportion of active element components, has poor uniformity, small porosity, and contains harmful heavy metal elements (such as lead, mercury, Heavy metal elements such as arsenic and cadmium) pose a certain safety risk to the human body. Moreover, the natural jade material itself has dense crystal growth and is relatively inert. The effective calcium, magnesium, silicon, zinc and other trace elements are not easily released, and the release of common biological beauty effects such as zinc, selenium, copper, and manganese is not controlled. For example, the content and release rate of zinc and selenium in natural jade materials are relatively low, which cannot meet the requirements for exerting ideal biological activity and beauty effects. Another example is that natural jade from some origins contains excessive amounts of elements such as manganese and copper, which are more cytotoxic and affect the safety of use.

### SUMMARY OF THE INVENTION

The purpose of the invention is to overcome the problems in the prior art and to provide a process control for the corresponding design and assembly of the composition and molecular structure of the material according to the actual demand. Finally meet the practical application needs of the assembly of jade activation materials and assembly synthesis methods.

One aspect of the present invention provides an assembled jade activation material for beauty. The assembled jade activation material consists of the 55-59% tetraethyl orthosilicate, 4-8% calcium nitrate tetrahydrate, 26-30% magnesium nitrate hexahydrate, 1-8% zinc nitrate, 0-3% yeast selenium, 0-1% manganese nitrate and 0-1% copper nitrate, and the sum of the mole percentages of each component is 100%.

Another aspect of the present invention provides a method for assembling and synthesizing assembled jade activation material for beauty. The material is assembled and synthesized by the sol-gel method, and the 55-59% tetraethyl orthosilicate, 4-8% calcium nitrate tetrahydrate, 26-30% magnesium nitrate hexahydrate, 1-8% zinc nitrate, 0-3% yeast selenium, 0-1% manganese nitrate and 0-1% copper nitrate were obtained by molar ratio, and the sum of the mole percentages of each component is 100%.

According to an embodiment of the assembled synthesis method of the assembled jade activated material used for beauty,

A. Pre-hydrolyze ltetraethyl orthosilicate under the catalysis of nitric acid solution;

B. The remaining raw materials are sequentially added and fully stirred, and the dispersant is added and stirred to form a clear and uniform sol;

C. The sol is aged to form a gel, and the dry gel powder obtained by drying, grinding and sieving the gel is calcined at a high temperature to obtain the assembled jade activation material.

On the other hand, the invention provides an assembled jade activation material for beauty, which is prepared by the assembly synthesis method of the assembled jade activation material for beauty.

According to an embodiment of the assembled jade activated material for beauty of the present invention, the assembled jade activated material has a specific surface area of 79.6-132.5 m²·g⁻¹, a porosity of 43%-56%, and at least includes silicon, calcium, magnesium and zinc. Among them, the ion dissolution concentration of silicon is 35.26-54.97 μg·mL⁻¹, the dissolution concentration of calcium is 275.2-304.5 μg·mL⁻¹, the dissolution concentration of magnesium is 68.35-90.17 μg·mL⁻¹, and the dissolution concentration of zinc is 8.47-20.336 μg·mL⁻¹.

According to an embodiment of the assembled jade activated material used for beauty, the assembly jade activated material scavenging rate of superoxide anion over 89%, scavenging rate of hydroxyl radical over 70%, and inhibition rate of yolk peroxidation over 70%.

Another aspect of the present invention provides the application of the assembled jade activating material for beauty in the preparation of cosmetics, skin care products or medical beauty products.

The invention draws on the main components of natural jade materials that exert their effects, and synthesizes active jade materials through self-assembly by the sol-gel method, which uniquely increases the release of trace elements silicon, calcium, and magnesium ions, and adjusts the amount of trace elements according to demand and the introduction of some other elements that are beneficial to the skin, such as the introduction of zinc, selenium, copper, manganese and other elements, to give full play to the excellent effects of these ingredients on the skin, the proportion of ingredients is precisely controllable and the purpose of controlling the release rate of active ions is achieved. It is expected to add a new material that is safe and has excellent efficacy for the research and development of products in the field of medical beauty or cosmetics.

### BRIEF DESCRIPTION OF THE DRAWINGS

FIG. 1 shows the microstructure of the assembled jade activated materials used for beauty.

FIG. 2 shows the microstructure of natural jade material of comparative example 1.

FIG. 3 shows the microstructure of the assembled jade activated materials prepared by comparative example 2.

FIG. 4 shows the MTT cell staining photos of the assembled jade activated materials.

FIG. 5 shows the MTT cell staining photos of the assembled jade activated materials.

FIG. 6 shows the cell test fibroblast staining photos of the invention.

### DETAILED DESCRIPTION

All features disclosed in this specification, all disclosed methods or steps in the process, except for mutually exclusive features and/or steps, can be combined in any manner.

Unless specifically stated, any feature disclosed in this specification can be replaced by other equivalent or equivalent alternative features. That is, unless otherwise stated, each feature is just one example of a series of equivalent or similar features.

In order to play the role of jade materials in beauty more safely and effectively, the assembled jade activation material synthesized by the self-assembly technology of the present invention has the ability to design and tailor the composition and molecular structure of the material according to actual needs, thereby endowing the material with specific biological and physical and chemical characteristics, and ultimately meet the needs of practical applications and play a better role than natural jade materials.

According to an exemplary embodiment of the present invention, the assembled jade activation material is composed of 55-59% tetraethyl orthosilicate, 4-8% calcium nitrate tetrahydrate, 26-30% magnesium nitrate hexahydrate, 1-8% zinc nitrate, and 0-3% yeast selenium, 0-1% manganese nitrate and 0-1% copper nitrate, where the sum of the mole percentages of each component is 100%. Preferably, the sol-gel method is used to prepare the assembled jade activation material.

The following will describe the assembling and synthesizing method of the assembled jade activation material for beauty in the present invention.

According to an exemplary embodiment of the present invention, the assembled jade activation material is assembled and synthesized by a sol-gel method. The assembled jade activation material is composed of 55-59% tetraethyl orthosilicate, 4-8% calcium nitrate tetrahydrate, 26-30% magnesium nitrate hexahydrate, 1-8% zinc nitrate, and 0-3% yeast selenium, 0-1% manganese nitrate and 0-1% copper nitrate, where the sum of the mole percentages of each component is 100%.

The formula design of the assembled jade activation material of the present invention forms a nanoporous amorphous glass as a whole. The present invention draws on the preparation method of bioactive glass, but does not completely refer to the design of conventional bioactive glass preparation (silicon, calcium, sodium, phosphorus). Among them, the present invention selects the effective ion to select the ratio of tetraethyl orthosilicate of 55-59% in order to achieve the purpose of controlling good biological activity. If the addition ratio of tetraethyl orthosilicate is less than 55%, the composition of vitrified silicon during assembly and synthesis will be too low, and the drying process of gel formation is prone to salt precipitation, which will affect the uniformity of the material. If the addition ratio of tetraethyl orthosilicate is higher than 59%, it will affect the release of product biological activity and reduce the biological activity of the material.

As the main functional ions, the ratio of calcium, magnesium, and zinc ions must be strictly controlled. Too low calcium ions are not conducive to stimulating the expression of growth factors in cells, and it is not conducive to innovative repair and stimulating the expression of EGF and FGF growth factors; too high calcium ions (such as more than 8%) will significantly increase the ion release of the material. The pH value will have a negative impact on the application;

Magnesium is the main functional ion that affects the direction of anti-oxidation and beauty. If it is too low, it will affect the anti-oxidation and anti-inflammatory effects of the material. If it is too high, the effect of enhancing the effect will not be achieved.

In addition, the formula introduces 1-8% zinc nitrate, which can meet the conventional beauty effects to enhance skin immunity, enhance skin elasticity and inhibit skin oil secretion, but the proportion will exceed the skin's demand limit for zinc ions, and the material toxicity will increase.

In the silicate glass body formed by the present invention, [SiO₄]⁻ is a disorderly arrangement, and the M⁺ or M²⁺ metal cations outside the framework are evenly distributed in the cavity of the framework, and play a role in balancing the negative charges of non-bridging oxygen. The four elements of basic silicon, calcium, magnesium and zinc cooperate with each other to form a complete slow-release system material of silicate amorphous structure.

The control of the aforementioned proportion can control the release rate of various elements to perform their effects according to the requirements, which can coordinate and enhance skin anti-inflammatory, cell repair and skin anti-aging effects without reducing the toxicity and safety characteristics of the material.

In addition, the formula design introduces 0-3% yeast selenium, 0-1% manganese nitrate and 0-1% copper nitrate, so that the material not only has the effect of anti-oxidative stress, but also can enhance the anti-oxidation effect of the formula. However, if the amount is excessive, it will increase the cytotoxicity of the material and affect the biosafety of the material and reduce the repair and repair efficacy of the material. If the content is too low or not added, it will reduce the corresponding brightening skin color, lightening scars and acne marks, and anti-aging effects.

The invention draws on the preparation method of bioactive glass, and synthesizes jade activation material through self-assembly of sol-gel method. This method can not only increase the release of trace elements silicon, calcium, magnesium, zinc, copper, selenium, manganese ions, etc., but also adjust the amount and type of trace elements according to actual needs.

Different active ions have different beauty biological activities and effects. For example, calcium has the function of skin barrier repair and improvement of skin sensitivity symptoms; Magnesium has anti-oxidation, participates in skin metabolism, repairs damaged skin nerves after sun or after surgery, and relieves dermatitis and pain; Zinc has the function of accelerating and enhancing the regeneration of skin wound tissues, improving immune function, resisting and eliminating skin pathogens, inhibiting the growth of acne and dark sore, making the skin smooth and elastic; Copper can strengthen the metabolism of protein and nucleic acid and harmful free radicals, balance melanin, effectively anti-wrinkle and anti-aging, dispel spots and yellow, sunscreen, shrink pores, and make the skin white, tender and smooth; Selenium can resist aging, repair damage, enhance immunity, etc.; Manganese has the functions of resisting the damage caused by free radicals to the human body, participating in the synthesis of protein and vitamins, accelerating metabolism, and anti-aging; Silicon has biological activity and can help the skin resist external damage, aging, and cell metabolism disorders.

Specifically, the assembly synthesis method includes the following steps.

Step A:

The tetraethyl orthosilicate is pre-hydrolyzed under the catalysis of nitric acid solution.

Among them, the nitric acid solution is used as a pre-hydrolysis catalyst, the molar concentration is preferably 1-2 mol·L⁻¹, the pre-hydrolysis time is 20-60 minutes, and the molar ratio of tetraethyl orthosilicate to water is preferably 1:8-1:12.

If the pre-hydrolysis time is less than 20 minutes, the hydrolysis will be insufficient, and the alcohol gelation of the material will not be completely formed. On the contrary, if the time is too long, such as more than 60 minutes, the material will be completely hydrolyzed, which will affect the production efficiency.

Step B:

The remaining raw materials are added in sequence and fully stirred evenly, and then the dispersant is added to form a clear and uniform sol.

The dispersant used in the present invention is preferably at least one of polyvinyl alcohol, glycerol, glycerol glucose, polyethylene glycol-400, polyethylene glycol-600, polyethylene glycol-1000 and polyethylene glycol-2000 The amount of dispersant added is 0.005-0.025 g mL⁻¹, and the stirring time after adding the dispersant is 0.5-2 hours.

Among them, the amount of dispersant added can be calculated based on the volume of the previously dissolved liquid. The use of dispersant can make the material obtain good nano-voids. Too low the amount of dispersant will increase the bulk density and significantly reduce the porosity after the material is formed, which cannot achieve the required high specific surface area and uniform void structure; If the amount of dispersant is more than 0.025 g mL⁻¹ will easily cause the gap size to be different, which is not conducive to the uniformity of the material and the stability of the sustained ion release.

Step C:

The sol is aged to form a gel, and the dry gel powder prepared by drying, grinding and sieving the gel is calcined at a high temperature to obtain the assembled jade active material powder.

Preferably, in the aging step, the aging temperature is 60-90° C., and the aging time is 36-60 hours; in the drying step, the drying temperature is 60-85° C., and the drying time is 36-60 hours; In the calcination step, the calcination temperature is 680-920° C., and the calcination time is 1-2.5 hours.

For aging, if the temperature is lower than 60° C., the efficiency will be significantly reduced. If the temperature exceeds 90° C., the nitrogen oxides will decompose violently and affect the safety of preparation; For calcination, the temperature below 680° C. will affect the nitrogen oxides of the material removal efficiency. If the temperature exceeds 920° C., it will seriously affect the crystal development and integrity of the material. The material will completely crystallize to form a complete dense crystal and reduce the biological activity of the material.

The assembled jade activated material for beauty of the present invention is preferably prepared by the above-mentioned method.

Specifically, the specific surface area of the assembled jade activation material of the present invention is 79.6-132.5 m²·g⁻¹, its porosity is 43%-56%, the pH value of the ion extract is 7.8-9.6, and the hydroxyl radical scavenging rate is 70%-75%, and at least include silicon, calcium, magnesium, zinc and other elements. Among them, the ion dissolution concentration of silicon is 35.26-54.97 μg·mL⁻¹, the dissolution concentration of calcium is 275.2-304.5 μg·mL⁻¹, the dissolution concentration of magnesium is 68.35-90.17 μg·mL⁻¹, and the dissolution concentration of zinc is 8.47-20.33 μg·mL⁻¹.

The assembled jade activated material of the present invention can be used in the production of cosmetics, skin care products or medical beauty products, in order to obtain corresponding beauty and nourishing effects.

The above-mentioned assembled jade activated material was used for in vitro testing of cosmetic efficacy. The material has a scavenging rate of superoxide anion over 89%, a scavenging rate of hydroxyl radical over 70%, and an inhibition rate of yolk peroxidation over 70%. Under the same test conditions, the inhibition of superoxide anion and yolk peroxidation is equivalent to that of VC. The scavenging rate of hydroxyl radical is better than that of VC.

The assembled jade activated material prepared by self-assembly of the present invention has a good anti-aging effect, the skin elasticity change rate is 0.049-0.072%, which are all positive values; The superoxide anion free radical scavenging rate is 89.01-92.36%, which has a good antioxidant effect; Biological cell anti-inflammatory test can significantly reduce the expression of 1L-α mRNA and 1L-6 mRNA (P<0.01), and significantly reduce the expression of TNF-α mRNA (P<0.05), with excellent anti-inflammatory effects; It can stimulate the wound cells to produce FGF and EGF, thereby promoting the repair of skin wounds.

### 1. Example of Failed Screening of Key Mix

According to the designed ratio and the alike method, specifically, tetraethyl orthosilicate was pre-hydrolyzed under the catalysis of a 2 mol·L⁻¹ nitric acid solution for 30 minutes, and then other raw materials were added in sequence and stirred to dissolve. Then 0.01 g·mL⁻¹ glycerol and 0.02 g·mL⁻¹ PEG-400 were added and stirred for 1 hour to form a clear and uniform sol. A gel was formed by aging at 70° C. for 48 hours, and then the gel was vacuum dried at 70° C. for 48 hours, giving dry gel powder through ground and sieved. Finally, the dry gel powder was calcined at 700° C. for 2 hours to obtain a formulation test example.

### Screening Test Example 1

60% tetraethyl orthosilicate, 10% calcium nitrate tetrahydrate, 22% magnesium nitrate hexahydrate, 8% zinc nitrate were weighed by mole, respectively.

### Screening Test Example 2

53% tetraethyl orthosilicate, 13% calcium nitrate tetrahydrate, 26% magnesium nitrate hexahydrate, 8% zinc nitrate were weighed by mole, respectively.

### Screening Test Example 3

55% tetraethyl orthosilicate, 9% calcium nitrate tetrahydrate, 24% magnesium nitrate hexahydrate, 7% zinc nitrate, 3% yeast selenium, 1% manganese nitrate and 1% copper nitrate were weighed by mole, respectively.

### Screening Test Example 4

55% tetraethyl orthosilicate, 8% calcium nitrate tetrahydrate, 30% magnesium nitrate hexahydrate, 4% zinc nitrate, 1.5% manganese nitrate and 1.5% copper nitrate were weighed by mole, respectively.

### 2. Examples of Failed Screening of Key Process Parameters

The process parameters were selected with a formula of 59% tetraethyl orthosilicate, 8% calcium nitrate tetrahydrate, 30% magnesium nitrate hexahydrate, and 3% zinc nitrate. The percentage here is also the mole percentage.

The tetraethyl orthosilicate was pre-hydrolyzed under the catalysis of a 1.5 mol·L⁻¹ nitric acid solution for 50 minutes. and then other raw materials were added in sequence and stirred to dissolve. Then 0.015 g·mL⁻¹ polyvinyl alcohol and 0.025 g·mL⁻¹ PEG-1000 were added and stirred for 2 hours to form a clear and uniform sol. A gel was formed by aging at 90° C. for 36 hours, and then the gel was vacuum dried at X ° C. for 40 hours, giving dry gel powder through ground and sieved. Finally, the dry gel powder was calcined at Y ° C. for 1 hours to obtain a formulation test example.

### Screening Test Example 5

The above-mentioned parameter X was set to 100° C. and the gel was vacuum dried under this temperature condition. However, the nitrogen oxides in the gel were occurred violently decomposed, causing the experiment to fail.

### Screening Test Example 6

The above-mentioned parameter Y was set to 930° C. and the gel was calcined for 1 hour to obtain the assembled jade activation material under this temperature condition.

TABLE 1
Evaluation Form of Screening.

| | | | Evaluation of | |
|---|---|---|---|---|
| | Evaluation of material | | Biological | Acceptability |
| Example | performance | pH | activity | of material |
| Screening test | The poor ion dissolution | 7.7 | bad | Unacceptability |
| example 1 | and biological activity due | | | |
| | to high silicon content of | | | |
| | the materials | | | |
| Screening test | The poor materials | 9.8 | good | Unacceptability |
| example 2 | uniformity due to | | | |
| | salting-out was occurred | | | |
| | during the gel formation | | | |
| | process | | | |
| Screening test | The poor biocompatibility | 10.2 | good | Unacceptability |
| example 3 | in cosmetic applications | | | |
| | due to the high ion | | | |
| | dissolution-out in the | | | |
| | materials | | | |
| Screening test | The poor biocompatibility, | 9.2 | good | Unacceptability |
| example 4 | which because of the | | | |
| | dissolution-out of copper | | | |
| | ion exceeded the standard | | | |
| | of application | | | |
| Screening test | The experimental product | | | |
| example 5 | is yellowish brown because | | | |
| | nitrogen oxides decompose | — | — | Unacceptability |
| | violently during the | | | |
| | gelation of the material | | | |
| Screening test | The crystal growth of the | 7.7 | bad | Unacceptability |
| example 6 | material is complete and | | | |
| | compact through XRD and | | | |
| | SEM characterization, | | | |
| | resulting in poor biological | | | |
| | activity of the material | | | |

The results of each screening test example are shown in Table 1. All the screening test examples in the table are the proportions and process parameters that failed in the research and development process. The present invention is a specific technical solution summarized through optimized components, preferred ratios and preferred solutions based on the results of multiple failed experiments, and can produce corresponding technical effects.

### Example 1

Specifically, 55% tetraethyl orthosilicate, 8% calcium nitrate tetrahydrate, 26% magnesium nitrate hexahydrate, 8% zinc nitrate, 1% yeast selenium, 1% manganese nitrate, and 1% copper nitrate were weighed by mole. tetraethyl orthosilicate was pre-hydrolyzed under the catalysis of a 2 mol·L⁻¹ nitric acid solution for 30 minutes, and then other raw materials were added in sequence and stirred to dissolve. Then 0.01 g·mL⁻¹ glycerol and 0.02 g·mL⁻¹ PEG-400 were added and stirred for 1 hour to form a clear and uniform sol. A gel was formed by aging at 70° C. for 48 hours, and then the gel was vacuum dried at 70° C. for 48 hours, giving dry gel powder through ground and sieved. Finally, the dry gel powder was calcined at 700° C. for 2 hours to obtain the assembled jade activation material Example 1.

### Example 2

Specifically, 59% tetraethyl orthosilicate, 6% calcium nitrate tetrahydrate, 30% magnesium nitrate hexahydrate, 4% zinc nitrate, 0.5% yeast selenium, 0.5% manganese nitrate were weighed by mole. tetraethyl orthosilicate was pre-hydrolyzed under the catalysis of a 1 mol·L⁻¹ nitric acid solution for 40 minutes, and then other raw materials were added in sequence and stirred to dissolve. Then 0.015 g·mL⁻¹ PEG-600 was added and stirred for 1.5 hour to form a clear and uniform sol. A gel was formed by aging at 80° C. for 42 hours, and then the gel was vacuum dried at 80° C. for 36 hours, giving dry gel powder through ground and sieved. Finally, the dry gel powder was calcined at 800° C. for 1 hour to obtain the assembled jade activation material Example 2.

### Example 3

Specifically, 59% tetraethyl orthosilicate, 8% calcium nitrate tetrahydrate, 30% magnesium nitrate hexahydrate, 3% zinc nitrate were weighed by mole. tetraethyl orthosilicate was pre-hydrolyzed under the catalysis of a 1.5 mol·L⁻¹ nitric acid solution for 50 minutes, and then other raw materials were added in sequence and stirred to dissolve. Then 0.015 g·mL⁻¹ polyvinyl alcohol and 0.025 g·mL⁻¹ PEG-1000 were added and stirred for 2 hour to form a clear and uniform sol. A gel was formed by aging at 90° C. for 36 hours, and then the gel was vacuum dried at 60° C. for 40 hours, giving dry gel powder through ground and sieved. Finally, the dry gel powder was calcined at 920° C. for 1 hours to obtain the assembled jade activation material Example 3.

### Example 4

Specifically, 57% tetraethyl orthosilicate, 5% calcium nitrate tetrahydrate, 30% magnesium nitrate hexahydrate, 7% zinc nitrate and 1% manganese nitrate were weighed by mole. tetraethyl orthosilicate was pre-hydrolyzed under the catalysis of a 1.8 mol·L⁻¹ nitric acid solution for 60 minutes, and then other raw materials were added in sequence and stirred to dissolve. Then 0.01 g·mL⁻¹ PEG-2000 was added and stirred for 0.5 hour to form a clear and uniform sol. A gel was formed by aging at 60° C. for 60 hours, and then the gel was vacuum dried at 85° C. for 36 hours, giving dry gel powder through ground and sieved. Finally, the dry gel powder was calcined at 680° C. for 2.5 hours to obtain the assembled jade activation material Example 4.

### Example 5

Specifically, 56% tetraethyl orthosilicate, 4% calcium nitrate tetrahydrate, 30% magnesium nitrate hexahydrate, 7% zinc nitrate and 3% yeast selenium were weighed by mole. tetraethyl orthosilicate was pre-hydrolyzed under the catalysis of a 1.8 mol·L⁻¹ nitric acid solution for 60 minutes, and then other raw materials were added in sequence and stirred to dissolve. Then 0.02 g·mL⁻¹ glycerol glucose and 0.01 g·mL⁻¹ PEG-400 was added and stirred for 0.5 hour to form a clear and uniform sol. A gel was formed by aging at 60° C. for 60 hours, and then the gel was vacuum dried at 85° C. for 36 hours, giving dry gel powder through ground and sieved. Finally, the dry gel powder was calcined at 680° C. for 2.5 hours to obtain the assembled jade activation material Example 5.

Comparative example 1: Natural jade material

Comparative example 2: The artificial jade material was prepared by the fusion method. Analytically pure nitrate, yeast selenium and tetraethyl orthosilicate were used as raw materials. Specifically, 59% tetraethyl orthosilicate, 4% calcium nitrate tetrahydrate, 26% magnesium nitrate hexahydrate, 8% zinc nitrate, 1% copper nitrate, 1% yeast selenium and 1% manganese nitrate were weighed by mole and mixed evenly. The mixed materials were put into a platinum crucible and melted at 1300° C. for 1.5 h. After melting, the melted materials were poured into a graphite mold annealed at 500° C. in a muffle furnace. Finally, the artificial jade material was obtained through grinding and sieving.

Comparative example 3: The artificial jade material was prepared by the fusion method. Analytically pure nitrate, and tetraethyl orthosilicate were used as raw materials. Specifically, 59% tetraethyl orthosilicate, 8% calcium nitrate tetrahydrate, 36% magnesium nitrate hexahydrate, and 3% zinc nitrate were weighed by mole and mixed evenly. The mixed materials were put into a platinum crucible and melted at 1300° C. for 1.5 h. After melting, the melted materials were poured into a graphite mold annealed at 500° C. in a muffle furnace. Finally, the artificial jade material was obtained through grinding and sieving.

### Experimental Example 1: Specific Surface Area Determination

The specific surface area of the assembled jade activation material prepared by all examples of the present invention and the jade materials used for the comparison samples were determined by the Brunauer-Emmett-Teller (BET) method. The specific test results are shown in Table 2 below. The results show that the assembled jade activation material prepared by the method of the present invention has a larger specific surface area compared to the comparison example (natural jade materials and synthetic jade materials synthesized by melting).

TABLE 2
The specific surface area of example assembled
jade materials and experimental example jade materials.

| Samples | Specific surface area (m² · g⁻¹) |
|---|---|
| example 1 | 132.5 |
| example 2 | 105.7 |
| example 3 | 80.2 |
| example 4 | 79.6 |
| example 5 | 93.3 |
| Experimental Example 1 | 9.6 |
| Experimental Example 2 | 11.8 |
| Experimental Example 3 | 8.7 |

### Experimental Example 2: Porosity and Homogeneity

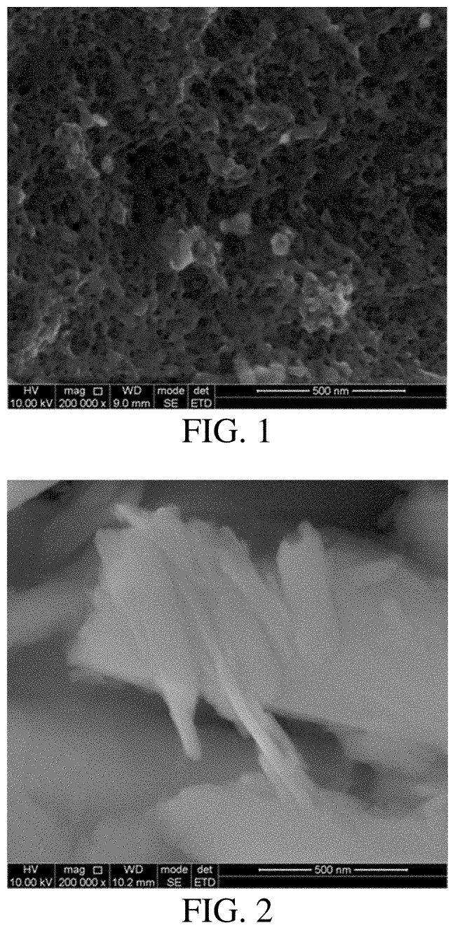

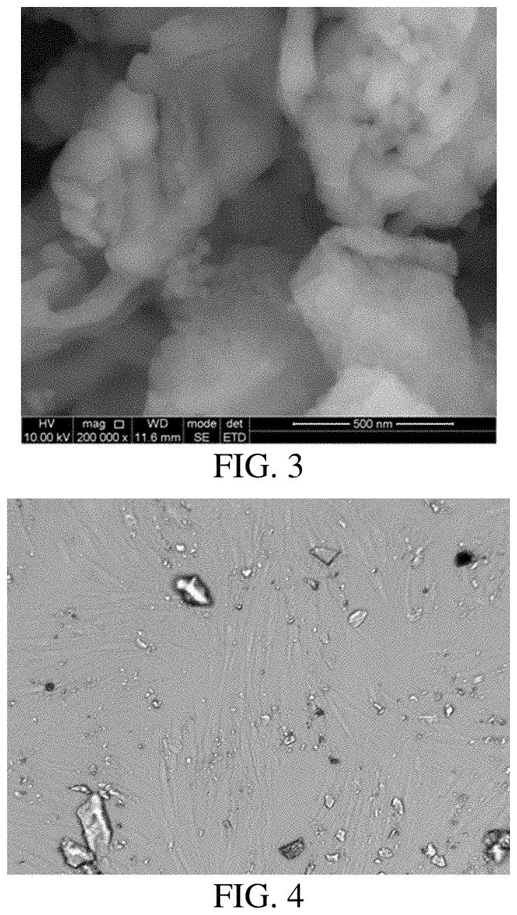

Characterization of the porosity and homogeneity of the assembled jade activation material prepared by all examples of the present invention and the jade materials used for the comparison samples by mass volume method and scanning electron microscope (SEM). The specific test results are shown in Table 3, FIG. 1, FIG. 2 and FIG. 3.

TABLE 3
True density and total porosity test
results of example assembled jade materials
and experimental example jade materials.

| | True | Total |
|---|---|---|
| Samples | Density (g · cm⁻³) | porosity (%) |
| Experimental Example 1 | 2.95 | 7.87 |
| Experimental Example 2 | 2.83 | 10.42 |
| Experimental Example 3 | 2.87 | 10.19 |
| example 1 | 1.94 | 56.35 |
| example 2 | 1.91 | 49.18 |
| example 3 | 1.98 | 43.46 |
| example 4 | 1.95 | 45.24 |
| example 5 | 1.90 | 47.33 |

Combining Table 3 with FIG. 1, FIG. 2 and FIG. 3, it can be seen that the assembled jade activation material prepared by the method of the present invention has better porosity and homogeneity compared to the comparison example (natural jade materials and synthetic jade materials synthesized by melting).

### Experimental Example 3: Safety and Stability

The heavy metal contents of the assembled jade activation material prepared by all examples of the present invention and the jade materials used for the comparison samples were determined by the method specified in the [symbol] Safety and Technical Standards for Cosmetics [symbol] , and the specific test results are shown in Table 4. The assembled jade activation material prepared by present invention contains very low amount of lead heavy metal, which meets the specification. Although the arsenic, cadmium and mercury in the natural jade material did not exceed the standard, its lead content exceeded the standard. Some heavy metals of lead were detected in the assembled jade activation material and synthetic jade materials, but were significantly lower than natural jade materials. Improper screening of natural jade materials will have certain safety risks.

TABLE 4
Heavy metal content determination results of example assembled jade materials and
experimental example jade materials.

| Samples | Pb (mg · kg⁻¹) | As (mg · kg⁻¹) | Cd (mg · kg⁻¹) | Hg (mg · kg⁻¹) |
|---|---|---|---|---|
| example 1 | Not Detected | Not Detected | Not Detected | Not Detected |
| example 2 | 1.31 | Not Detected | Not Detected | Not Detected |
| example 3 | 0.91 | Not Detected | Not Detected | Not Detected |
| example 4 | Not Detected | Not Detected | Not Detected | Not Detected |
| example 5 | 0.65 | Not Detected | Not Detected | Not Detected |
| Experimental Example 1 | 27.04 | 1.5 | 3.1 | 0.53 |
| Experimental Example 2 | 4.43 | Not Detected | Not Detected | Not Detected |
| Experimental Example 3 | 2.06 | Not Detected | Not Detected | Not Detected |

Natural jade materials of different origins, even from the same origin, have different compositions and contain heavy metals such as lead, mercury, cadmium, arsenic, etc., leading to potential problems with the stability and safety of natural jade materials. On the contrary, the self-assembled synthetic assembled jade activation material of the present invention can control the type of added elements and the amount of added elements according to the demand, while the adulteration of heavy metal elements can be effectively avoided from the raw material.

### Experimental Example 4

Experimental release and biological activity evaluation of trace elements of calcium, magnesium, silicon, zinc and manganese. The ion dissolution of the assembled jade activation material prepared by each embodiment of the present invention and the jade material of each pair of proportions was illustrated by ion dissolution experiments, as shown in Table 5, which shows that the concentration of each trace element dissolved in the assembled jade activation material prepared by the present invention is significantly higher compared with the jade material of each pair of proportions, increasing the role of trace elements.

TABLE 5
Ion dissolution of example assembled jade materials and experimental example jade materials.

| | C (Ca²⁺) | C (Mg²⁺) | C (Si⁴⁺) | C (Zn²⁺) | C (Mn⁴⁺) | Ion leaching | Bioactivity |
|---|---|---|---|---|---|---|---|
| Samples | (μg · mL⁻¹) | (μg · mL⁻¹) | (μg · mL⁻¹) | (μg · mL⁻¹) | (μg · mL⁻¹) | solution pH | evaluation |
| example 1 | 304.5 | 90.17 | 54.97 | 20.33 | 8.26 | 9.6 | Very good, |
| | | | | | | | acceptable |
| example 2 | 283.3 | 85.12 | 48.64 | 11.47 | 2.15 | 9.1 | Good, |
| | | | | | | | acceptable |
| example 3 | 275.2 | 68.35 | 35.26 | 9.61 | — | 7.8 | Normal, |
| | | | | | | | acceptable |
| example 4 | 278.36 | 77.93 | 37.22 | 18.59 | 10.34 | 8.7 | Good, |
| | | | | | | | acceptable |
| example 5 | 280.61 | 80.91 | 41.21 | 19.38 | — | 8.9 | Good, |
| | | | | | | | acceptable |
| Experimental | 14.58 | 10.32 | 20.39 | 1.26 | 0.93 | 7.5 | Bad, |
| Example 1 | | | | | | | Unacceptable |
| Experimental | 11.31 | 16.73 | 10.46 | 1.10 | 0.34 | 7.1 | Bad, |
| Example 2 | | | | | | | Unacceptable |
| Experimental | 4.62 | 8.55 | 3.75 | 0.14 | — | 6.8 | Bad, |
| Example 3 | | | | | | | Unacceptable |

### Experimental Example 5: Anti-Aging Effect

Anti-aging efficacy evaluation test was conducted with a skin elasticity tester. Specifically, the creams obtained by adding 5% of the assembled jade activation material obtained in Examples 1-5 of the present invention and the jade materials in proportion 1-3 to the same base material were prepared, respectively. The base material cream was used as a blank group. The creams with different jade materials added and the blank creams were applied on the fresh porcine skin evenly. The anti-aging efficacy of the jade material was evaluated by examining the change in the elasticity condition of the porcine skin before and after the application of the different creams.

The test environment for anti-aging efficacy evaluation test requires constant temperature and humidity, 14-16° C. and 30-40% humidity. Fresh porcine skin was selected for the test, and the porcine skin was treated into two 50 mm×50 mm test areas. The porcine skin were placed into two petri dishes separately to test the initial elasticity value of the porcine skin, different creams were applied to the porcine skin and left flat in the test environment for 2 h, and the elasticity value of the two pieces of porcine skin was measured by a skin elasticity tester. The rate of change in the elasticity of the porcine skin before and after the application of the cream was calculated using the following equation:

$$W = \frac{N - {N}_{0}}{{N}_{0}} \times 100 \%$$

In the equation, W is the rate of change of skin elasticity, unit is %; N is the test value of skin elasticity after the specimen is treated with porcine skin; No is the initial value of skin elasticity test.

Criteria for selecting the porcine skin model: the skin of small suckling porcines was selected because the skin of small suckling porcines has a high similarity to human skin. Specifically, fresh small suckling porcine skin was taken and the subcutaneous tissue was removed for the test, and the results are shown in Table 6.

TABLE 6
The rate of change of elasticity of porcine
skin after applying different creams.

| Samples | Elastic rate of change (%) |
|---|---|
| example 1 | 0.072 |
| example 2 | 0.065 |
| example 3 | 0.049 |
| example 4 | 0.058 |
| example 5 | 0.053 |
| Experimental Example 1 | 0.016 |
| Experimental Example 2 | 0.029 |
| Experimental Example 3 | 0.021 |
| Control example | −0.12 |

As can be seen from the table, the rate of change of elasticity of porcine skin in the blank group was negative 0.12, indicating that the elasticity of porcine skin in the blank group decreased. The rate of change of elasticity of porcine skin in the sample groups of jade activating material prepared by adding the present invention and jade synthetic material prepared by the comparative example were both positive, which indicated that the elasticity of porcine skin increased. The effect of the sample group of jade activating material prepared by the present invention was the most obvious, and the samples of comparative example 2 and comparative example 3 with the addition of efficacious elements also had an enhanced effect on the elasticity value, thus indicating that the cream with the addition of jade activating material prepared by the present invention assembled has a good anti-aging effect.

### Experimental Example 6: Antioxidant Effect

The antioxidant efficacy of the assembled jade activation material prepared by each embodiment of the present invention was demonstrated by the scavenging rate of superoxide anion and hydroxyl radical, as well as the peroxidation inhibition rate of yolk lipids in comparison with the natural jade material of comparative example 1, the test results are shown in Table 7. Comparing the data in the table, it is obvious that the antioxidant efficacy of the assembled jade activation material made by the present invention is optimal.

TABLE 7
Antioxidant effect of example assembled jade materials
and experimental example jade materials.
Experimental Example 7: Anti-inflammatory effect.

| | | | Scavenging | Scavenging | Peroxidation |
|---|---|---|---|---|---|
| | | | rate of | rate of | inhibition rate |
| | Concentration | Number | superoxide | hydroxyl | of yolk lipids |
| Samples | (mg · mL⁻¹) | (N) | anion (%) | radical (%) | (%) |
| Example 1 | 2 | 3 | 92.68 | 75.86 | 79.57 |
| Example 2 | 2 | 3 | 91.03 | 74.09 | 75.32 |
| Example 3 | 2 | 3 | 89.97 | 72.05 | 72.89 |
| Example 4 | 2 | 3 | 91.55 | 72.91 | 73.24 |
| Example 5 | 2 | 3 | 89.04 | 73.58 | 75.12 |
| Experimental | 2 | 3 | 79.42 | 12.03 | 64.85 |
| Example 1 | 2 | 3 | 92.39 | 49.83 | 70.94 |
| VC | | | | | |

### Experimental Example 7: Anti-Inflammatory Effect

Group A consisted of natural jade material of comparative example 1; Group B consisted of assembled jade activation material of Example 1. Statistical results of the RCR test on common inflammatory factors in human skin cells showed that:

Groups A and B: IL-1α mRNA expression was significantly reduced in human cells in group A (P<0.05, significant difference); IL-1α mRNA expression was significantly reduced in human cells in group B (P<0.01, highly significant difference).

Groups A and B: IL-6 mRNA expression was significantly reduced in human cells in group A (P<0.05, significant difference); IL-6 mRNA expression was significantly reduced in human cells in group B (P<0.01, highly significant difference).

Groups A and B: TNF-α mRNA expression was significantly reduced in human cells of both groups A and B, with significant differences (P<0.05).

It is clear that the assembled jade activation material of the present invention has more significant anti-inflammatory efficacy compared with the natural jade material of comparative example 1.

### Experimental Example 8: Skin Trauma Damage Repair

The principal growth factors associated with skin wound repair involve fibroblast growth factors (FGF), epidermal growth factor (EGF), and so on.

Group A consisted of natural jade material in proportion 1; Group B consisted of assembled jade activating material in Example 1. Wounds were made on the back of SD rats, and creams with natural jade material (group A), assembled jade activation material (group B), and blank creams without any efficacious ingredients (blank control group) were applied around the wounds periodically, respectively. The changes of EGF and FGF expression in the surrounding tissues of the trauma were detected by RT-PCR. The statistical results of RT-PCR for animal experiments are shown in Table 8.

TABLE 8
Expression of mRN EGF/mRN bFGF gene at various times in different groups (x ± s,
n = 5, note: Compared with the blank group, *P < 0.05).
Expression of mRNA EGF

| Group | Two days | Severn days | Fourteen days |
|---|---|---|---|
| A | 6.14 ± 1.18* | 19.62 ± 2.05* | 8.52 ± 02.08* |
| B | 6.85 ± 1.23* | 21.6 ± 1.95* | 8.88 ± 01.77* |
| blank control group | 4.52 ± 1.17 | 4.42 ± 1.18 | 4.44 ± 01.17 |

Expression of mRNA bFGF

| Group | Two days | Seven days | Fourteen days |
|---|---|---|---|
| A | 4.98 ± 1.05* | 18.45 ± 1.75* | 7.79 ± 0.35* |
| B | 6.04 ± 1.01* | 21.21 ± 1.24* | 8.61 ± 1.12* |
| blank control group | 3.41 ± 1.01 | 3.42 ± 1.03 | 3.53 ± 1.11 |

The rats were executed after the application of the different creams at 2, 7 and 14 days, and the RNA was extracted from the edges of the wounds and then performed RT-PCR experiments. The results showed that the gene transcription levels of EGF and FGF in both groups A and B were significantly higher than those in the blank control group after the total skin defect on the back of rats, and group B was better than group A. The difference between group AB and the control group was statistically significant (P<0.05, significant difference).

It can be seen that the blank cream with the addition of the assembled jade activation material of the present invention and the natural jade material both have the effect of promoting the secretion of FGF and EGF, and the effect is better with the addition of the assembled jade activation material. This indicates that the assembled jade activating material of the present invention has a greater effect of stimulating the production of FGF and EGF by the trauma cells and thus promoting the repair of skin trauma.

### Experimental Example 9: Safety of Materials (Inhibition Rate Test of Human Fibroblasts—MTT Test)

Scheme 1: Group A is the natural jade material of comparative example 1; Group B is the assembled jade activation material of Example 1, and human fibroblast inhibition rate MTT test was performed. The experimental results are shown in Table 9, which shows that the inhibition rate of human fibroblasts in group B is much lower than that in group A with the same concentration, indicating that the assembled jade activation material of the present invention has better safety.

TABLE 9
MTT assay of human fibroblast inhibition
rate for different samples.

| Group | Concentration | Number of tests | Average inhibition rate (%) |
|---|---|---|---|
| A | 5 mg · mL⁻¹ | 5 | 61.46 |
| | l mg · mL⁻¹ | 5 | 56.59 |
| | 0.5 mg · mL⁻¹ | 5 | 57.03 |
| B | 5 mg · mL⁻¹ | 5 | 31.75 |
| | 1 mg · mL⁻¹ | 5 | 10.87 |
| | 0.5 mg · mL⁻¹ | 5 | −0.97 |

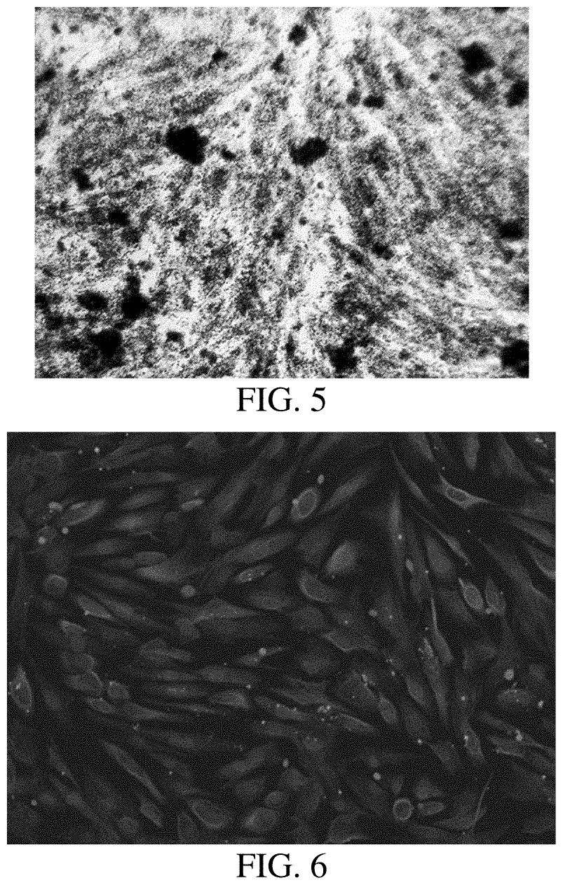

As can be seen from Table 9, the assembled jade activation material of Example 1 of Group B was significantly lower than the natural jade material of Group A comparative example 1 on human fibroinhibition at different exposure concentrations, indicating that the cytotoxicity of Example 1 was significantly lower in all cases. FIG. 4 illustrates MTT cell staining photographs of the assembled jade activating material of the present invention and FIG. 5 illustrates MTT cell staining photographs of the assembled jade activating material of the present invention, demonstrating the effect on cell morphology upon material exposure. Referring to FIG. 6 illustrates the fibroblast cell staining photos used in the cell assay of the present invention.

Scheme 2: Group A is the natural jade material of comparative example 1; Group B is the assembled jade activation material of Example 1. The relative growth rate (RGR) of the jade material was calculated by MTT method in accordance with GB/T16175-2008 standard when cultured on mouse fibroblast L929 cells to determine the cytotoxicity of the material, and the experimental results are shown in Table 10.

TABLE 10
Relative value-added rate of fibroblasts
in different samples (MTT method).

| | | | | Levels of |
|---|---|---|---|---|
| | Group | Levels of | Group | cytotoxic |
| | A/PGR (%) | cytotoxicity | B/PGR (%) | response |
| Additional | 66.14 | 2 | 115.31 | 0 |
| samples for 3 days | | | | |
| Additional | 75.61 | 2 | 96.63 | 1 |
| samples for 5 days | | | | |
| Additional | 85.01 | 1 | 85.12 | 1 |
| samples for 7 days | | | | |

Note:
Level 0 means no cytotoxicity, level 1 means very slight cytotoxicity

From Table 10, it can be seen that the Relative reproduction rate PGR of fibroblasts decreased step by step with the extension of culture time, and the PGR values of group B were higher than those of group A. The cytotoxic response level of group B was between 0 and 1, which can be considered as non-toxic to fibroblasts; the cytotoxic response level of group A was 1 to 2, which was very slightly toxic to fibroblasts. Thus, it can be seen that the assembled jade activation material prepared by self-assembly of the present invention is less cytotoxic and safer compared with natural jade material.

In summary, the self-assembled jade activation material of the present invention has a wide and stable source of basic raw materials compared with natural jade materials used in cosmetics; the original formulation design of the assembled jade activation material is homogeneous, precisely controlled and stable in composition, and free from other harmful impurities such as heavy metals; the specific surface area and biological activity of the material are improved, which enhances the safety, anti-inflammatory, skin repair and anti-aging effects of the assembled jade material. It increases the release of trace elements such as calcium, magnesium, silicon ions, etc., and the amount and type of trace elements can be adjusted according to the actual demand; it further improves and perfects the skin care efficacy of the raw materials, and has stronger and more comprehensive skin care efficacy.

The present invention is not limited to the specific embodiments described above. The invention extends to any new feature, any new combination disclosed in this specification, as well as to any new method, any step of the process and any new combination disclosed.

1. The assembled jade activation material for beauty, characterized in that the material is assembled and synthesized by 55-59% tetraethyl orthosilicate, 4-8% calcium nitrate tetrahydrate, 26-30% magnesium nitrate hexahydrate, 1-8% zinc nitrate, 0-3% yeast selenium, 0-1% manganese nitrate and 0-1% copper nitrate, and the sum of the mole percentages of each component is 100%

2. According to claim 1, the assembled jade activated material for beauty is characterized in that the specific surface area of this material is 79.6-132.5 m²·g⁻¹, the porosity is 43%-56%, and it includes at least silicon, calcium, magnesium and zinc elements. Among them, the dissolution concentration of silicon is 35.26-54.97 μg·mL⁻¹, the dissolution concentration of calcium is 275.2-304.5 μg·mL⁻¹, the dissolution concentration of magnesium is 68.35-90.17 μg·mL⁻¹, and the dissolution concentration of zinc is 8.47-20.33 μg·mL⁻¹. The assembled jade activation material has a scavenging rate of superoxide anion over 89%, a scavenging rate of hydroxyl radical over 70%, and a inhibition rate of yolk peroxidation over 70%.

3. The method for assembling and synthesizing the assembled jade activation material for beauty is characterized in that the material is assembled and synthesized by the sol-gel method, and the 55-59% tetratetraethyl orthosilicate, 4-8% calcium nitrate tetrahydrate, 26-30% magnesium nitrate hexahydrate, 1-8% zinc nitrate, 0-3% yeast selenium, 0-1% manganese nitrate and 0-1% copper nitrate were obtained by molar ratio, and the sum of the mole percentages of each component is 100%.

4. According to claim 3, the assembling and synthesizing method of the assembled jade activation material for beauty is characterized in that it comprises the following steps:
A. Pre-hydrolyze tetraethyl orthosilicate under the catalysis of nitric acid solution;
B. The remaining raw materials are sequentially added and fully stirred, and the dispersant is added and stirred to form a clear and uniform sol;
C. The sol is aged to form a gel, and the dry gel powder obtained by drying, grinding and sieving the gel is calcined at a high temperature to obtain the assembled jade activation material.

5. According to claim 4, the assembling and synthesizing method of the assembled jade activation material for beauty is characterized in that the molar concentration of the nitric acid solution is 1-2 mol·L⁻¹, and the pre-hydrolysis time is 20-60 minutes, wherein the molar ratio of tetraethyl orthosilicate to water is 1:8-1:12.

6. According to claim 4, the assembling and synthesizing method of the assembled jade activation material for beauty is characterized in that the dispersant is at least one of polyvinyl alcohol, glycerol, glycerol glucose, polyethylene glycol-400, polyethylene glycol-600, polyethylene glycol-1000 and polyethylene glycol-2000, the addition amount of the dispersant is 0.005-0.025 g·mL⁻¹, and the stirring time after adding the dispersant is 0.5-2 hours.

7. According to claim 4, the assembling and synthesizing method of the assembled jade activation material for beauty is characterized in that the aging temperature is 60-90° C., and the aging time is 36-60 hours; the drying temperature is 60-85° C., and the drying time is 36-60 hours; the calcination temperature is 680-920° C., and the calcination time is 1-2.5 hours.

8. The assembled jade activation material for beauty is characterized in that it needs to be prepared by the assembly synthesis method described in claim 3.

9. According to claim 8, the assembled jade activation material for beauty is characterized in that the material has a specific surface area of 79.6-132.5 m²·g⁻¹, a porosity of 43%-56%, and at least includes silicon, calcium, magnesium, and zinc, among which the dissolution concentration of silicon ion is 35.26-54.97 μg·mL⁻¹, the dissolution concentration of calcium ion is 275.2-304.5 μg·mL⁻¹, the dissolution concentration of magnesium ion is 68.35-90.17 μg·mL⁻¹, and the dissolution concentration of magnesium is 68.35-90.17 μg·mL⁻¹. The ionic dissolution concentration is 8.47-20.33 μg·mL⁻¹. The assembled jade activation material has a scavenging rate of superoxide anion over 89%, a scavenging rate of hydroxyl radical over 70%, and a inhibition rate of yolk peroxidation over 70%.

10. As claimed in claim 1, the assembled jade activation material for beauty can be used in the preparation of cosmetics, skin care products and medical beauty products.
